(19) United States
(12) Patent Application Publication
(10) Pub. No.: US 2020/0286631 A1
(43) Pub. Date: Sep. 10, 2020

(54) AUTOMATIC AUGMENTED SECURE MEDICAL COMMUNICATION

(71) Applicant: PIONEERRX, LLC, Shreveport, LA (US)

(72) Inventors: Stephen CULVER, Shreveport, LA (US); Wyatt DICKSON, Irving, TX (US); Loren FISHER, Irving, TX (US); Jeff KEY, Colleyville, TX (US); Paul CARRIG, Shreveport, LA (US)

(21) Appl. No.: 16/809,532

(22) Filed: Mar. 4, 2020

Related U.S. Application Data
(60) Provisional application No. 62/813,298, filed on Mar. 4, 2019.

(51) Int. Cl. G16H 80/00; G06Q 10/10; G16H 10/60; H04L 29/06; G06K 19/06; H04N 1/44

(52) U.S. Cl. CPC G16H 80/00; G06Q 10/107; G16H 10/60; H04N 1/4413; G06K 19/06028; G06K 19/06037; G06K 19/0614; H04L 63/08

(57) ABSTRACT

The presently disclosed invention relates to methods secure automated augmented messaging systems comprising a pharmacy messaging system having a first processor, a first display, and a non-volatile first memory with instructions stored upon, a doctor messaging system having a first messaging unit having a second processor, a second display, and a non-volatile second memory with instructions stored thereupon, and a second messaging unit having a third processor, a third display, and a non-volatile third memory with instructions stored thereupon, and a redundant messaging system having a fourth processor, a fourth display, and a non-volatile fourth memory with instructions stored thereupon, wherein instructions stored on the respective memories cause the respective processors of the systems and units to cause the pharmacy messaging system to automatically send a redundant message in a second format to the second messaging unit when the first messaging system sends a first message to the first messaging unit in a first format.

### CROSS REFERENCE TO RELATED APPLICATIONS/PRIORITY

The present invention claims priority to U.S. Provisional Patent Application No. 62/813,298 filed Mar. 4, 2019, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### BACKGROUND

In the course of delivering patient care, patient care providers, such as pharmacies, must sometimes communicate patient information with other patient care providers, such as doctor's offices. Acceptable HIPAA methods include phone conversations, physical mail, and fax. With electronic medical records, there is a current government requirement that there must be “meaningful use” to receive Medicare funding. To do meaningful use, messages must be sent in a received in a HIPAA secure way called secure direct messaging. There is currently a standard accepted secure direct messaging method to exchange messages securely. When received the messages appear similar in format to email, and the addresses look very similar to email, though it is a different ecosystem.

Upon testing a secure direct message for pharmacies, the inventor discovered multiple technical issues. In some instances, the secure direct message was rejected because the sender was not on a doctor's office or organization's whitelist. In other situations, the doctor's office's system does not fully support the format. For example, if the secure direct message had an attachment, in some instances, the recipient's system may accept the sender's text part of the message, but reject the message attachment, or reject both because of the attachment. Further, if the doctor's office's system rejects all or part of the pharmacy's secure direct message, the pharmacy may also not learn the doctor never received all or part of the pharmacy's secure direct message. This has led many pharmacies and doctors to be reluctant to use secure direct messaging. This is a technical problem, requiring a technical solution, that has real world implications on patient health.

### SUMMARY

Wherefore, it is an object of an embodiment of the presently disclosed invention to overcome some or all of the above-mentioned shortcomings and drawbacks associated with the current technology. The presently disclosed invention is directed to methods and apparatuses that satisfy the above shortcomings and drawbacks.

The presently disclosed invention also relates to methods and secure automated augmented messaging systems comprising a first messaging system having a first processor, a first display, and a non-volatile first memory with instructions stored upon, a second messaging system having a first messaging unit, a second processor, a second display, and a non-volatile second memory with instructions stored thereupon, and a second messaging unit having a third processor, a third display, and a non-volatile third memory with instructions stored thereupon, and a third messaging device having a fourth processor, a fourth display, and a non-volatile fourth memory with instructions stored thereupon, wherein instructions stored on the respective memories cause the respective processors of the devices and units to cause the pharmacy messaging device to automatically send a redundant message in a second format to the second messaging unit when the pharmacy messaging device sends a first message to the first messaging unit.

The presently disclosed invention further relates to systems and methods of automatically augmenting secure messaging comprising sending a first message as a secure message from an first messaging system to a first messaging device of a second messaging system and automatically sending a redundant message second messaging device of the second messaging system, wherein the redundant message includes the first message, a unique identifier, and a plurality of selectables. According to further embodiments the second messaging unit is a fax machine. According to further embodiments the redundant message is in the format of a fax message. According to further embodiments the first message is in the format of a secure direct message. According to further embodiments the selectables include items to be checked indicating that the first message was not received via the first messaging device, the first message was received via the first messaging device, and/or confirmation of the correctness of the address that the first message was sent to According to further embodiments the unique identifier contains one of an alphanumeric code, a substantially non-alphanumeric symbol, and both an alphanumeric code and a substantially non-alphanumeric symbol. According to further embodiments the non-alphanumeric symbol is a bar code.

The presently disclosed invention relates to methods secure automated augmented messaging systems comprising a pharmacy messaging system having a first processor, a first display, and a non-volatile first memory with instructions stored upon, a doctor messaging system having a first messaging unit having a second processor, a second display, and a non-volatile second memory with instructions stored thereupon, and a second messaging unit having a third processor, a third display, and a non-volatile third memory with instructions stored thereupon, and a redundant messaging system having a fourth processor, a fourth display, and a non-volatile fourth memory with instructions stored thereupon, wherein instructions stored on the respective memories cause the respective processors of the systems and units to cause the pharmacy messaging system to automatically send a redundant message in a second format to the second messaging unit when the first messaging system sends a first message to the first messaging unit in a first format. According to a further embodiment, the second messaging unit is a fax machine. According to a further embodiment, the second format is a fax message. According to a further embodiment, the first format is a secure direct message. According to a further embodiment, the second message includes substantially all of the information as the first message, and also includes a unique identifier that is perceivable to a human. According to a further embodiment, the instructions stored on the respective memories cause the respective processors of the systems and units to automatically scan a return message sent by the second message unit to the redundant messaging system for a unique identifier. According to a further embodiment, the second format is one of email, text, virtual fax, and Interactive Voice Response (IVR). According to a further embodiment, a hyperlink is sent via email or text with the redundant message, which when the doctor clicks on directs the doctor to a website hosted by one of the pharmacy system and redundant system, and when the doctor enter a unique doctor ID and password when prompted on the website, the doctor is directed to a screen where the doctor may directly type out a reply message. According to a further embodiment, first message is automatically assigned an ID, and the pharmacy messaging system, the doctor messaging system, the first, second and any reply message, and a unique identifier are automatically associated with the ID.

The disclosed invention is further related to systems and methods of automatically augmenting secure messaging comprising sending a first message as a secure message from a first messaging system to a first messaging device of a second messaging system, and automatically generating and sending a redundant message to a second messaging device of the second messaging system, wherein the redundant message includes the first message, a unique identifier, and a plurality of selectable; and wherein the first messaging system includes a first processor, a first display, and a non-volatile first memory with instructions stored upon, and the second messaging system includes a first messaging unit having a second processor, a second display, and a non-volatile second memory with instructions stored thereupon, and a second messaging unit having a third processor, a third display, and a non-volatile third memory with instructions stored thereupon. According to a further embodiment, the second messaging unit is a fax machine. According to a further embodiment, the redundant message is in the format of a fax message. According to a further embodiment, the first message is in the format of a secure direct message. According to a further embodiment, the selectables include a plurality of items to be checked indicating that the first message was not received via the first messaging unit, the first message was received via the first messaging unit, and confirmation of the correctness of the address that the first message was sent to. According to a further embodiment, the unique identifier contains one of an alphanumeric code, a substantially non-alphanumeric symbol, and both an alphanumeric code and a substantially non-alphanumeric symbol. According to a further embodiment, the non-alphanumeric symbol is a bar code. According to a further embodiment, the non-alphanumeric symbol is one of a Aztec Code, MaxiCode, ShotCode, PDF417, QR code, Qode, and High Capacity Color Barcode. According to a further embodiment, the method further comprises the steps of a third messaging system receiving a response message from the second messaging unit, associating the response message with the unique identifier, and forwarding the response message in the first format to the first messaging system, wherein the third messaging system has a fourth processor, and a non-volatile fourth memory with instructions stored thereupon. According to a further embodiment, the method further comprises the first messaging server continues to automatically generate and send redundant messages to the second messaging unit when the first messaging server sends subsequent messages as secure messages from the first messaging system to a first messaging unit until the first messaging server receives a return secure message from the first messaging unit.

The presently disclosed invention further relates to systems and methods of automatically augmenting secure medical messaging comprising sending a first message as a secure direct message from a pharmacy messaging system to a first messaging device of a doctor messaging system, and automatically generating and sending a fax message to a fax machine of the second messaging system, the fax message includes the first message, a unique identifier, and a plurality of selectable; a redundant messaging system receiving a response fax message from the fax machine and associating the response fax message with the unique identifier, and forwarding the response fax message as a secure direct message to the pharmacy messaging system, and the pharmacy messaging server continuing to automatically generate and send redundant fax messages to the fax machine when the pharmacy messaging server sends subsequent messages as secure direct messages from the pharmacy messaging system to a first messaging unit until the pharmacy messaging server receives a return secure direct message from the first messaging unit, wherein the selectables include a plurality of items to be checked indicating that the first message was not received via the first messaging unit, the first message was received via the first messaging unit, and confirmation of the correctness of the address that the first message was sent to, the unique identifier contains an alphanumeric code and a bar code, the pharmacy messaging system includes a first processor, a first display, and a non-volatile first memory with instructions stored upon, the doctor messaging system includes a first messaging unit having a second processor, a second display, and a non-volatile second memory with instructions stored thereupon, and a fax machine; and the redundant messaging system has a fourth processor, and a non-volatile fourth memory with instructions stored thereupon.

Various objects, features, aspects, and advantages of the presently disclosed invention will become more apparent from the following detailed description of preferred embodiments of the invention, along with the accompanying drawings in which like numerals represent like components. The presently disclosed invention may address one or more of the problems and deficiencies of the current technology discussed above. However, it is contemplated that the presently disclosed invention may prove useful in addressing other problems and deficiencies in a number of technical areas. Therefore, the presently disclosed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the presently disclosed invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The presently disclosed invention will now be described, by way of example, with reference to the accompanying drawings in which:

FIG. 1 is a general schematic diagram of a first embodiment of the disclosed automatic augmented secure medical messaging process according to the disclosed invention;

FIG. 2 is a flowchart of a second embodiment of the automatic augmented secure medical messaging process according to the disclosed invention similar to the first embodiment;

FIG. 3 is a flowchart of a further embodiment of the automatic augmented secure medical messaging process utilizing secure direct messages as the preferred method of FIG. 2 and fax messages as the alternative method of FIG. 2;

FIG. 4 is a fax message that could be sent in step 3.7 of the process depicted in FIG. 3, for example;

FIG. 5 is a fax message that could be sent in step 3.9 of the process depicted in FIG. 3, for example;

FIG. 6 is a schematic diagram of an embodiment of a doctor messaging system, a pharmacy messaging system, or redundancy messaging system according to the disclosed invention; and

### DETAILED DESCRIPTION

The presently disclosed invention will be understood by reference to the following detailed description, which should be read in conjunction with the appended drawings. It is to be appreciated that the following detailed description of various embodiments is by way of example only and is not meant to limit, in any way, the scope of the presently disclosed invention. In the summary above, in the following detailed description, in the claims below, and in the accompanying drawings, reference is made to particular features (including method steps) of the presently disclosed invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the presently disclosed invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

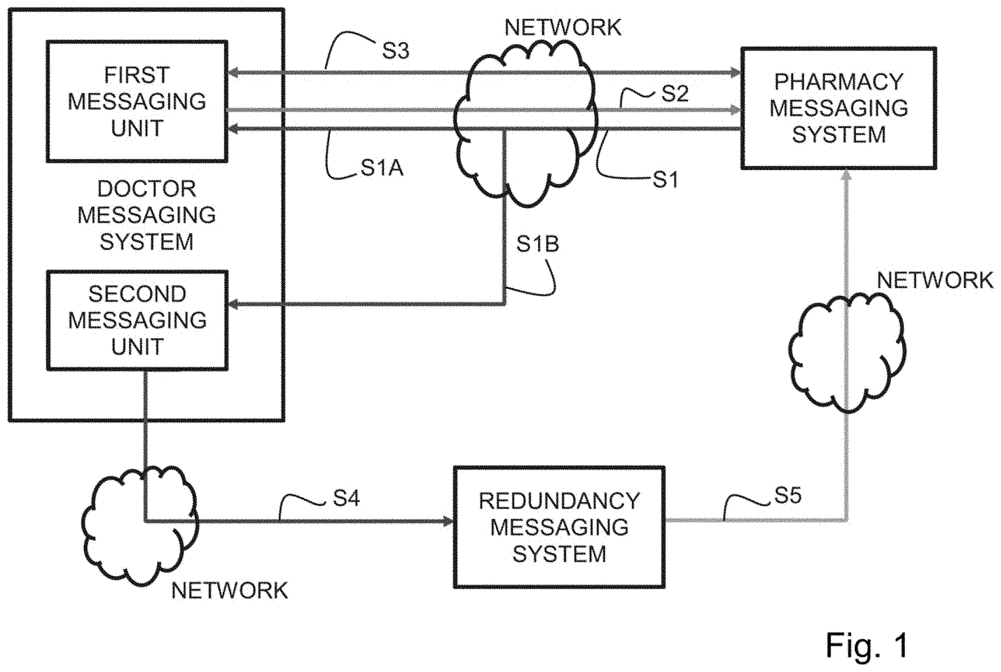

Turning now to FIG. 1, a brief description concerning the various components of the presently disclosed invention will now be briefly discussed. FIG. 1 provides a general overview of the automatic augmented secure medical messaging system disclosed herein. As can be seen in this embodiment, the automatic augmented secure medical communication system includes a first messaging system, a second messaging system, and a third messaging system, respectively displayed as the pharmacy messaging system, the doctor messaging system, and the redundancy messaging system in this embodiment. In this embodiment, the process may be initiated by a pharmacy worker sending a first message to a doctor that contains protected health information and thus is to be sent as a secure direct message. In a first step S1, the pharmacy worker composes and sends a first message as a secure direct message to the doctor at the doctor's messaging system. The message is assigned an ID, and the sender, recipient, message, and a unique identifier is associated with the ID. In the first step, the pharmacy messaging system may automatically send through a network both a first message to via a preferred communication method or first messaging unit of the doctor messaging system S1A and a redundant message to an alternative communication method or second messaging unit of the doctor messaging system S1B. The first messaging unit is preferably a secure messaging system, such as a HIPAA compliant direct messaging system. The second messaging unit may be a physical fax machine. Alternative second messaging units may be email, texts, virtual fax, and Interactive Voice Response (IVR), including HIPAA secured compliant versions thereof such as hyperlink sent via email or text that the doctor could click on and go to a website and enter a unique doctor ID and password, and then be directed to a screen where the doctor could, for example, directly type out a second message, as described below, and select one or more selectables, as described below. In a preferred embodiment, the redundant message would arrive as a fax message. The redundant message will preferably include the first message, a statement indicating that the first message was also sent via direct message to a specific direct message address, one or more unique identifiers, and one or more selectables. In the embodiment shown, a preferable first unique identifier is a bar code and a preferably second unique identifier is and alpha-numeric code. Other unique identifiers could be 2-D matrix codes such as Aztec Code, MaxiCode, ShotCode, PDF417, QR code, Qode, and High Capacity Color Barcode, for example. The selectables could include a plurality of statements and/or questions such as, “I received a direct message at the address indicated above”, “The indicated address is correct, but I did not receive a message”, “The indicated address is incorrect” and a space for writing in the correct address. Additionally, if an attachment was sent with the direct message, a statement indicating that an attachment was sent with this message via Direct Message could be included, and one or more selectables could be included such as, “I received the direct message and attachment at the indicated address”, “I received the direct message BUT NOT the attachment at the indicated address,” and “I did not receive the direct message or the attachment.” The selectables can have methods for selection, such as adjacent check boxes, or be numbered, or have other ways that a doctor can indicate which one or ones of the selectables is appropriate for the specific situation.

If the doctor did receive the first message in the first messaging unit as a secure direct message, then in step S2, the doctor can reply back through a network from the doctor messaging system a second message as a direct message to the pharmacy messaging system. The pharmacy messaging system would then receive and display the second message. The pharmacy messaging system could then note that the first message was successfully received by the first messaging unit, and then in step S3 allow further direct messages through a network from the pharmacy messaging system to be directly to the first messaging unit and not also the second messaging unit.

If the doctor elected to mark a selectable or provide any other data on the redundant message in creating the second message, then, in step S4, the doctor could then fax the marked up second message back to an indicated fax number. This second message would transmit through a network to a redundant messaging system. The message would be received and scanned for the unique identifier. In the case of a bar code unique identifier, the redundant messaging system could process the bar code image and, determine the ID associated with the bar code, and then associate the message associated with the ID. Then, the selectables portion of the second message, and any other new markings made by the doctor, could be automatically be copied and pasted into a third message image that, in step S5, could be sent to the pharmacy messaging server through a network as a reply to the first message. If there is any writing on the second message, the writing could be transcribed into typed text either automatically by the redundant messaging system, and/or by a human worker, and/or the automatically by the redundant messaging system and proofed by the human worker. In one embodiment, the functions of the redundant messaging server are performed by the pharmacy messaging server, and/or the human worker could work for a third company.

Turning next to FIG. 1, this figure is a flowchart of a second embodiment of the automatic augmented secure medical messaging process according to the disclosed invention, with steps S1.1 to S1.41, which mirrors many parts of the first embodiment above. In step S1.1 a sender, such as a pharmacy, creates a message on a first messaging system through a preferred communication method such as a pharmacy messaging system. In step S1.1 the sender creates a message through a preferred communication method. In step S1.3 the message is assigned a unique identifier. The system then determines if the recipient, such as a doctor's office, has a preferred communication method or not. In step S1.11, if the recipient does have a preferred communication method the system then determines if the recipient has confirmed or has not confirmed the preferred communication method. If, in step S1.21, the recipient has confirmed the preferred communication method the system moves to step S1.19 where the message is sent through the preferred communication method. Returning to step S1.11 if the recipient as unconfirmed preferred communication method, step S1.23, then in step S1.13 the recipient receives the message from both the preferred and the alternative communication methods. Next, in step S1.9 the recipient responds to the message via the preferred or the alternative communication method. Returning to step S1.5, if the recipient does not have a preferred communication method, in step S1.7 the recipient receives the message from alternative communication method. Next system moves to step S1.9.

After step S1.9, where the recipient responds to the message via preferred or alternate communication method, the system determines if the response message is received via the preferred or alternative communication method. If, as in step S1.17, the response message is received via preferred communication method, then the system queries if the recipient has an unconfirmed preferred communication method or does not have preferred communication method. If as in step S1.29, the recipient does not have a preferred communication method, then the system moves to step S1.37, where the user sets the recipient's preferred communication method pending confirmation. Returning to step S1.17, if, as in step 1.27, the recipient has an unconfirmed preferred communication method, the system moves to step S1.35, and the recipient's ability to communicate using the preferred method is confirmed.

Returning to step S1.9, if the response message is received via alternative communication methods, as in step S1.15, then, as in step S1.25, the recipients' response is matched to the original message automatically or via user input, noting the unique identifier assigned in step S1.3. They system then inquiries if the recipient has an unconfirmed preferred communication method or does not have a preferred communication method. If, as in step S1.31, the recipient has an unconfirmed preferred communication method, then the system moves to step S1.39, where the user confirms the recipient is able to communicate using the preferred method. Alternatively, if, as in step S1.33, the recipient does not have a preferred communication method, then the system moves to step S1.41, where the user sets the recipient's preferred communication method pending confirmation.

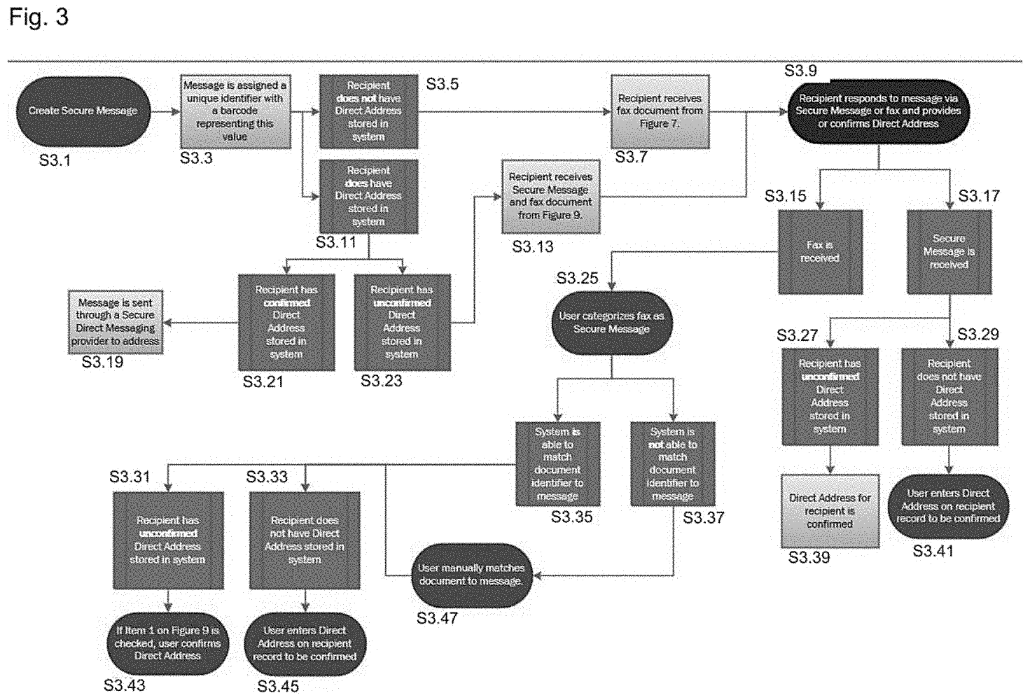

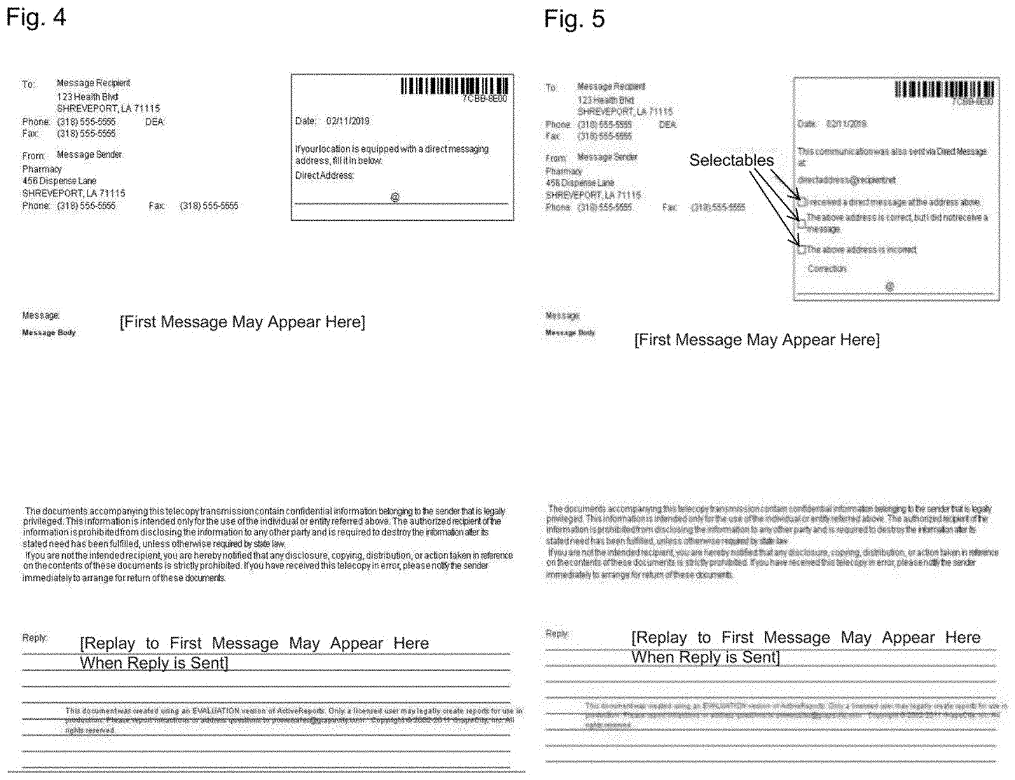

Turning next to FIG. 3, a flowchart of a further embodiment of the automatic augmented secure medical messaging process as described in FIG. 1 is shown, where the preferred communication method is a secure direct message and the alternative communication method is a fax machine, with steps S3.1 to S3.45. FIG. 4 is a fax message that could be sent in step S3.7 of the process depicted in FIG. 3. FIG. 5 is a fax message that could be sent in step S3.9 of the process depicted in FIG. 3, for example. As will be described, FIGS. 4 and 5 are largely the same, though FIG. 5 has the addition of selectables. Different selectables could be included in FIG. 4 though.

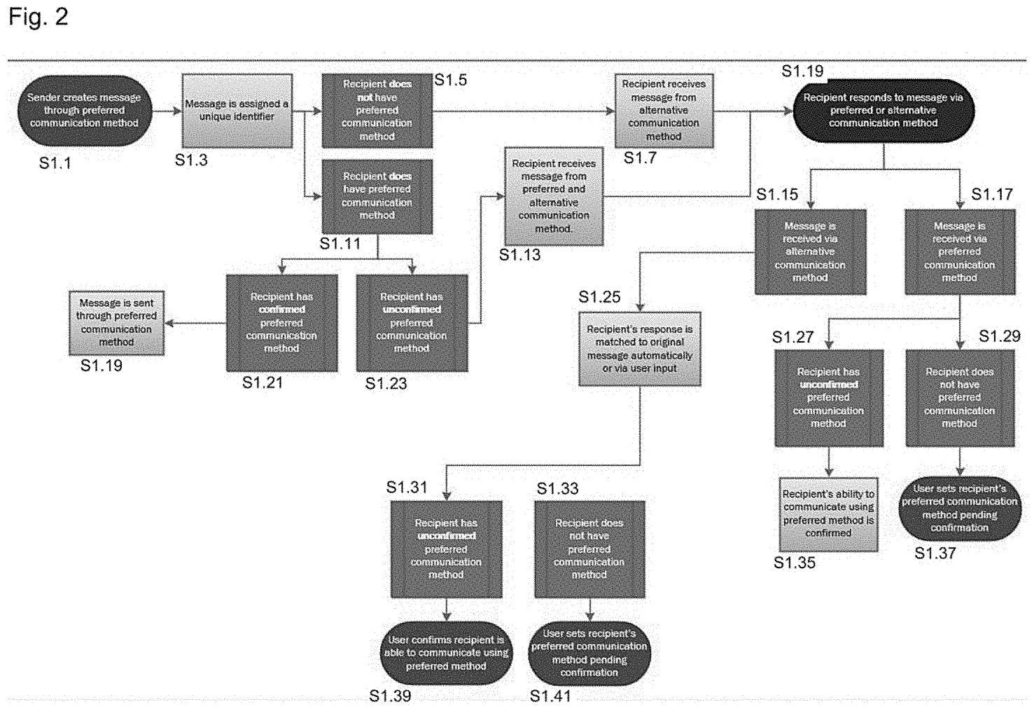

Discussing FIG. 3 the fax method embodiment of FIG. 2, in step S3.1 the user creates a secure message. In step S3.3 the message is assigned a unique identifier with a barcode representing this unique identifier value. Next the system queries if the recipient does or does not have a direct address stored in the system. The direct address being a secure direct message address, for example. If as in step S3.11 the recipient does have a direct address stored in the system, the system next queries if the recipient has confirmed the direct address or if the direct address stored in the system is unconfirmed. If as in step S3.21, the recipient has confirmed the direct address to in system, then the message is sent through a secure direct messaging provider to the address, as in 3.19. Returning to step S3.11, if the recipient has an unconfirmed direct address stored in system, step S3.23, then the recipient receives a both a secure message and a fax document from FIG. 5, in step S3.13. Then, in step S3.9, the recipient responds by sending a message via secure message or fax and provides or confirms direct address. Returning to step S3.5, if the recipient does not have a direct address stored in the system, the recipient receives a fax document, such as that shown in FIG. 4 in step S3.7, and then the system moves to step S3.9. The fax document can have an enrolment location where the recipient can enter a secure message address for further messages.

At step S3.9, after the recipient responds to the message via secure message or fax and provides or confirms a direct address, the system queries if a fax is received or if a secure message is received as the reply message. If, as in step S3.17, a secure message is received, then the system queries if the recipient has an unconcerned direct address stored in the system or if the recipient does not have a direct address stored in the system. If as in step S3.29 the recipient does not have a direct address stored in the system, the user enters direct address on recipients record to be confirmed in step S3.41. Returning to step S3.17, if the recipient has an unconfirmed direct address stored in the system, as in step S3.27, a direct address for the recipient is confirmed in step S3.39. Returning to step S3.9, if a fax is received as the reply message, as in step S3.15, then the user categorizes the fax as a secure message in step S3.25. Next, the system queries if it is able to automatically match the document identifier to the message, by optical character recognition or other program scanning or evaluating the document, or if the system is not able to match the document identifier to the message. If the system is not able to match the document identifier to the message, step S3.37, then the user manually matches the document to the message in step 3.47. Next, the system queries if the recipient has an unconfirmed direct address stored in the system or if the recipient does not have a direct address sorting system. If, as in step S3.31, the recipient has an unconfirmed direct address stored in the system, then in step S3.43 the system or user checks if any of the selectables in FIG. 5 are checked, and if so the user confirms direct address, and if not, the user does not confirm direct address. Returning to step S3.47, if the recipient does not have a direct address stored in the system, in step S3.33, the user enters a direct address on the recipient record to be confirmed, in step S3.45. Returning to step S3.25, if the system is able to match the document identifier to the message, in step S3.35, then the system queries if the recipient has an unconfirmed direct address stored in the system, step S3.31 or if the recipient does not have a direct address stored in the system, step S3.33, and continues as above.

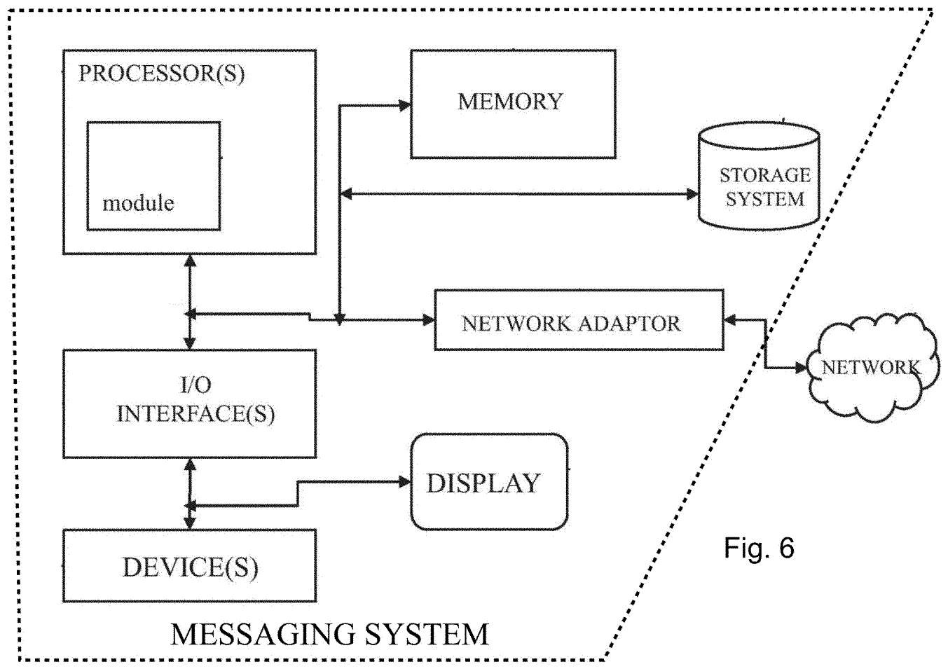

Turning now to FIG. 6, this drawing illustrates a schematic of an example computer or processing system of the first, the second, and the third messaging system, or the doctor messaging system, pharmacy messaging system, and redundancy messaging system, which may implement the disclosed methods in one embodiment of the present disclosure. The computer system is only one example of a suitable processing system and is not intended to suggest any limitation as to the scope of use or functionality of embodiments of the methodology described herein. The processing system shown may be operational with numerous other general purpose or special purpose computing system environments or configurations. Examples of well-known computing systems, environments, and/or configurations that may be suitable for use with the processing system shown in FIG. 6 may include, but are not limited to, personal computer systems, server computer systems, thin clients, thick clients, handheld or laptop devices, mobile devices, smart phones, tablet devices, multiprocessor systems, microprocessor-based systems, set top boxes, programmable consumer electronics, network PCs, minicomputer systems, mainframe computer systems, printers, faxes, and distributed cloud computing environments that include any of the above systems or devices, and the like.

The computer system may be described in the general context of computer system executable instructions, such as program modules, being executed by a computer system. Generally, program modules may include routines, programs, objects, components, logic, data structures, and so on that perform particular tasks or implement particular abstract data types. The computer system may be practiced in distributed cloud computing environments where tasks are performed by remote processing devices that are linked through a communications network. In a distributed cloud computing environment, program modules may be located in both local and remote computer system storage media including memory storage devices.

The components of computer system may include, but are not limited to, one or more processors or processing units, a system memory, and a bus that couples various system components including system memory to processor. The processor may include a module that performs the methods described herein. The module may be programmed into the integrated circuits of the processor, or loaded from memory, storage device, or network or combinations thereof.

Bus may represent one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system may include a variety of computer system readable media. Such media may be any available media that is accessible by computer system, and it may include both volatile and non-volatile media, removable and non-removable media.

System memory can include computer system readable media in the form of volatile memory, such as random access memory (RAM) and/or cache memory or others. Computer system may further include other removable/non-removable, volatile/non-volatile computer system storage media. By way of example only, storage system can be provided for reading from and writing to a non-removable, non-volatile magnetic media (e.g., a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus by one or more data media interfaces.

Computer system may also communicate with one or more external devices such as a keyboard, a pointing device, a display, etc.; one or more devices that enable a user to interact with computer system; and/or any devices (e.g., network card, modem, etc.) that enable computer system to communicate with one or more other computing devices. Such communication can occur via Input/Output (I/O) interfaces. Still yet, computer system can communicate with one or more networks such as a local area network (LAN), a general wide area network (WAN), and/or a public network (e.g., the Internet) via network adapter. As depicted, network adapter communicates with the other components of computer system via bus. It should be understood that although not shown, other hardware and/or software components could be used in conjunction with computer system. Examples include, but are not limited to: microcode, device drivers, redundant processing units, external disk drive arrays, RAID systems, tape drives, and data archival storage systems, etc.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements, if any, in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore I/we claim:

1. A secure automated augmented messaging system comprising
a pharmacy messaging system having
a first processor,
a first display, and
a non-volatile first memory with instructions stored upon,
a doctor messaging system having
a first messaging unit having
a second processor,
a second display, and
a non-volatile second memory with instructions stored thereupon, and
a second messaging unit having
a third processor,
a third display, and
a non-volatile third memory with instructions stored thereupon, and
a redundant messaging system having
a fourth processor,
a fourth display, and
a non-volatile fourth memory with instructions stored thereupon,
wherein instructions stored on the respective memories cause the respective processors of the systems and units to cause the pharmacy messaging system to automatically send a redundant message in a second format to the second messaging unit when the first messaging system sends a first message to the first messaging unit in a first format.

2. The system of claim 1 wherein the second messaging unit is a fax machine.

3. The system of claim 1 wherein the second format is a fax message.

4. The system of claim 1 wherein the first format is a secure direct message.

5. The system of claim 1 wherein the second message includes substantially all of the information as the first message, and also includes a unique identifier that is perceivable to a human.

6. The system of claim 1 wherein the instructions stored on the respective memories cause the respective processors of the systems and units to automatically scan a return message sent by the second message unit to the redundant messaging system for a unique identifier.

7. The system of claim 1 wherein the second format is one of email, text, virtual fax, and Interactive Voice Response (IVR).

8. The system of claim 1 wherein a hyperlink is sent via email or text with the redundant message, which when the doctor clicks on directs the doctor to a website hosted by one of the pharmacy system and redundant system, and when the doctor enter a unique doctor ID and password when prompted on the website, the doctor is directed to a screen where the doctor may directly type out a reply message.

9. The system of claim 1, wherein first message is automatically assigned an ID, and the pharmacy messaging system, the doctor messaging system, the first, second and any reply message, and a unique identifier are automatically associated with the ID.

10. A method of automatically augmenting secure messaging comprising:
sending a first message as a secure message from a first messaging system to a first messaging device of a second messaging system, and
automatically generating and sending a redundant message to a second messaging device of the second messaging system,
wherein the redundant message includes the first message, a unique identifier, and a plurality of selectable; and
wherein the first messaging system includes
a first processor,
a first display, and
a non-volatile first memory with instructions stored upon, and
the second messaging system includes
a first messaging unit having
a second processor,
a second display, and
a non-volatile second memory with instructions stored thereupon, and
a second messaging unit having
a third processor,
a third display, and
a non-volatile third memory with instructions stored thereupon

11. The method of claim 10 wherein the second messaging unit is a fax machine.

12. The method of claim 10 wherein the redundant message is in the format of a fax message.

13. The method of claim 10 wherein the first message is in the format of a secure direct message.

14. The method of claim 10 wherein the selectables include a plurality of items to be checked indicating that
the first message was not received via the first messaging unit,
the first message was received via the first messaging unit, and
confirmation of the correctness of the address that the first message was sent to.

15. The method of claim 10 wherein the unique identifier contains one of an alphanumeric code, a substantially non-alphanumeric symbol, and both an alphanumeric code and a substantially non-alphanumeric symbol.

16. The method of claim 15 wherein the non-alphanumeric symbol is a bar code.

17. The method of claim 15 wherein the non-alphanumeric symbol is one of a Aztec Code, MaxiCode, ShotCode, PDF417, QR code, Qode, and High Capacity Color Barcode.

18. The method of claim 10 further comprising the steps of a third messaging system receiving a response message from the second messaging unit, associating the response message with the unique identifier, and forwarding the response message in the first format to the first messaging system,
wherein the third messaging system has
a fourth processor, and
a non-volatile fourth memory with instructions stored thereupon.

19. The method of claim 10 further comprising the first messaging server continues to automatically generate and send redundant messages to the second messaging unit when the first messaging server sends subsequent messages as secure messages from the first messaging system to a first messaging unit until the first messaging server receives a return secure message from the first messaging unit.

20. A method of automatically augmenting secure medical messaging comprising:
sending a first message as a secure direct message from a pharmacy messaging system to a first messaging device of a doctor messaging system,
automatically generating and sending a fax message to a fax machine of the second messaging system, the fax message includes the first message, a unique identifier, and a plurality of selectable;
a redundant messaging system receiving a response fax message from the fax machine and associating the response fax message with the unique identifier, and forwarding the response fax message as a secure direct message to the pharmacy messaging system, and
the pharmacy messaging server continuing to automatically generate and send redundant fax messages to the fax machine when the pharmacy messaging server sends subsequent messages as secure direct messages from the pharmacy messaging system to a first messaging unit until the pharmacy messaging server receives a return secure direct message from the first messaging unit;
wherein
the selectables include a plurality of items to be checked indicating that
the first message was not received via the first messaging unit,
the first message was received via the first messaging unit, and
confirmation of the correctness of the address that the first message was sent to,
the unique identifier contains an alphanumeric code and a bar code,
the pharmacy messaging system includes
a first processor,
a first display, and
a non-volatile first memory with instructions stored upon,
the doctor messaging system includes
a first messaging unit having
a second processor,
a second display, and
a non-volatile second memory with instructions stored thereupon, and
a fax machine; and
the redundant messaging system has
a fourth processor, and
a non-volatile fourth memory with instructions stored thereupon.
